SEQUENCE LISTING (5 sequences)

SEQ ID NO: 1 - DNA, 21 bp, artificial sequence
  primer
cagtgctctagacgtgctagt

SEQ ID NO: 2 - DNA, 25 bp, artificial sequence
  primer
actagcacgtctagagcactgaaaa

SEQ ID NO: 3 - DNA, 46 bp, artificial sequence
  primer
cagtgctctagacgtgctagtactagcacgtctagagcactgaaaa

SEQ ID NO: 4 - DNA, 46 bp, artificial sequence; identical to SEQ ID NO: 3
  primer

SEQ ID NO: 5 - DNA, 22 bp, artificial sequence
  primer
tcagaggttggactaggtgatc

(19) United States
(12) Patent Application Publication
(10) Pub. No.: US 2008/0254516 A1
(43) Pub. Date: Oct. 16, 2008

(54) Method of Isolating Nucleic Acid Targets

(76) Inventors: Judith St. John, Littleton, CO (US); Thomas W. Quinn, Westminster, CO (US)

Correspondence Address: SHERIDAN ROSS PC, 1560 BROADWAY, SUITE 1200, DENVER, CO, 80202, US

(21) Appl. No.: 10/591,051

(22) PCT Filed: Mar. 1, 2005
(86) PCT No.: PCT/US05/06448

§ 371 Date: Jul. 13, 2007

Related U.S. Application Data
(60) Provisional application No. 60/548,769, filed on Feb. 27, 2004.

(51) Int. Cl. C12P 19/34; C07H 21/00

(52) U.S. Cl. 435/91.2; 536/25.4; 435/91.1; 536/23.1; 536/25.33

(57) ABSTRACT

The invention provides efficient methods of isolating specific nucleic acid targets to obtain information from target nucleic acid sequences in a relatively short time period. DNA or cDNA is enzymatically digested into smaller fragments, double-stranded DNA linkers are added onto the ends of the DNA fragments to flank each fragment with a known DNA sequence. The fragments are mixed with an oligonucleotide probe that is bound to a marker and contains a conserved nucleic acid sequence of interest. The fragments that hybridize to the probe through nucleotide base pair complementation become indirectly connected to the marker. These target fragments are captured using a capture agent that specifically recognizes the marker and treated to prevent non-specific binding. Captured fragments are typically cloned prior to sequencing. The captured fragments may also be amplified using PCR to increase the efficiency of the cloning.

### CROSS REFERENCE TO RELATED APPLICATION

This application is a national stage application under 35 U.S.C. 371 of PCT Application No. PCT/US2005/006448 having an international filing date of Feb. 28, 2005, which designated the United States, which PCT application claimed the benefit of U.S. Provisional Application Ser. No. 60/548,769, filed Feb. 27, 2004, the entire disclosure of which are hereby incorporated herein by reference.

### FIELD OF THE INVENTION

The invention resides in the field of molecular biology and specifically within techniques of isolating nucleic acid molecules of interest.

### BACKGROUND OF THE INVENTION

The goal of many projects involving molecular biology is to isolate a specific nucleic acid target that may lie within a very large genome. This target might be a certain gene that causes cancer, an area that controls the activity of an adjacent gene, a transposable element within the genome, regions of the DNA that help to make individual identifications through “DNA fingerprints,” an RNA transcript of a particular gene and the like. Such nucleic acid targets are typically pursued by biotechnology companies and academic research laboratories.

Earlier approaches to isolate specific targets involved searching through a large number of pieces of a fragmented genome that had been packaged within bacteriophage genomes or bacterial plasmids. The search required considerable time, and the manipulation of living bacteria, including infection with viral particles, required a certain level of expertise. In the mid-1980's, the invention of the polymerase chain reaction (PCR), often allowed an alternative approach that did not require passing DNA through living bacteria. However, this approach requires knowledge of the DNA sequences that flank the area of interest, something that is often unknown.

One very common target for isolation is a type of DNA sequence called a “microsatellite.” Microsatellites are short tandem repeats of simple sequence from 1 to 6 base pairs long. An example of a 2-base microsatellite would be the sequence “CACACACA”; and a 3-base microsatellite would be “CATCATCAT.” Microsatellites are highly mutable and as a result, there are typically many different alleles within a population. This makes it possible to distinguish between different individuals according to the subset of alleles that they carry within their genomes. By looking at many such loci, it is possible to “fingerprint” target organisms. This is one of the main methods used in human identification by the forensics community. It is also used extensively in conservation genetics and has recently been applied to studies of mutation rates in vertebrates from polluted areas. Because such studies require information from several microsatellite loci, and because previously identified microsatellites are rare in most organisms, methods have been developed to increase the efficiency of the original isolation and characterization of microsatellites. One method attempts to fragment genomic DNA from the organism of interest and selectively concentrate those fragments that contain microsatellite DNA. This type of procedure is called “enrichment.” These enrichment procedures can be cumbersome, often resulting in the co-isolation of high fractions of nucleic acid sequences of little or no interest. Therefore, there is a need for an efficient method of isolating target nucleic acid sequences from genomic DNA in a relatively short time period.

### BRIEF DESCRIPTION OF THE DRAWINGS

FIG. 1 shows a schematic diagram of the preparation of DNA fragments prior to hybridization in a preferred embodiment of the present invention

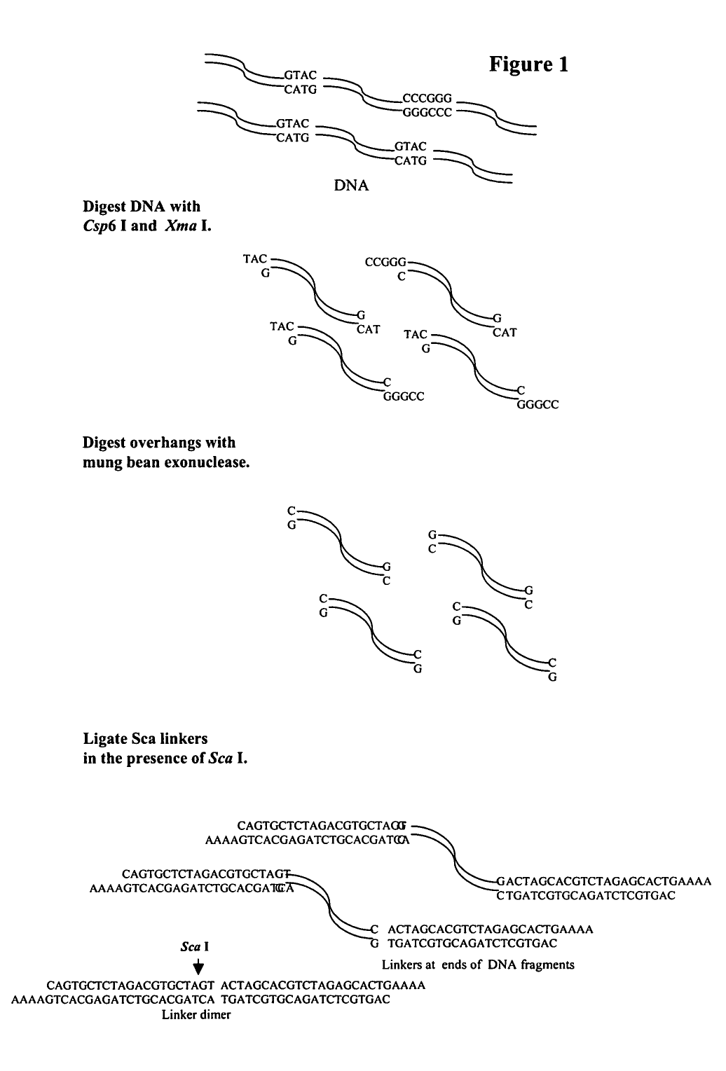

FIG. 2 shows a schematic diagram of the hybridization and capture of target nucleic acid fragments using one embodiment of the present invention.

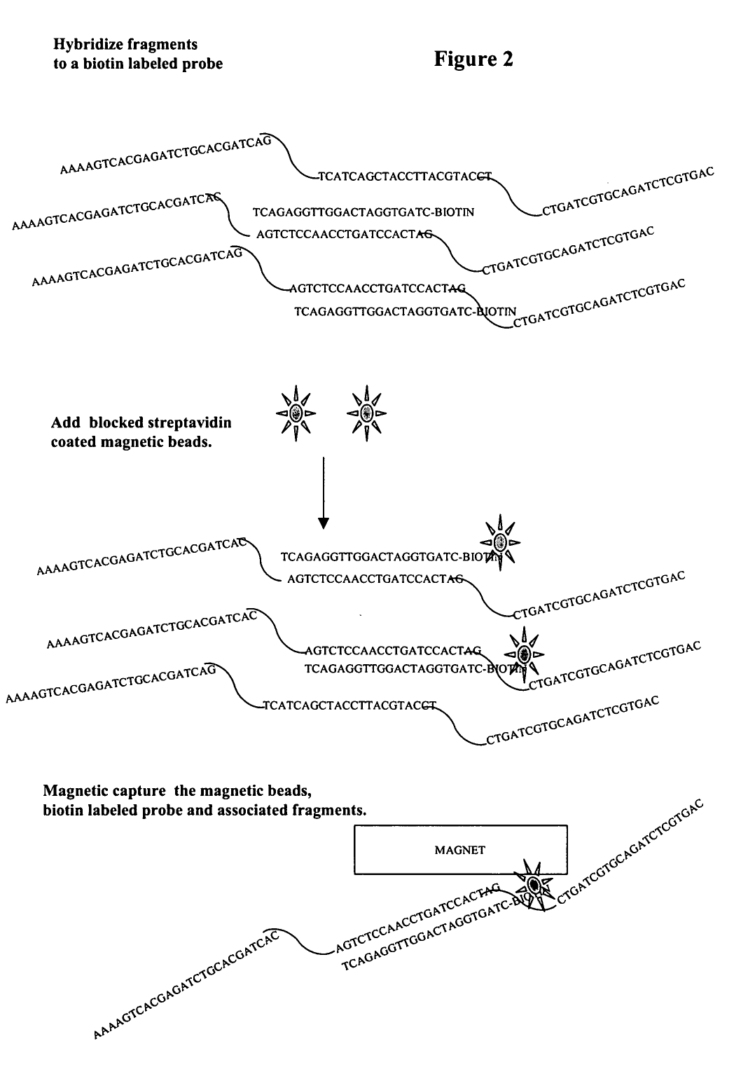

FIG. 3 shows a schematic diagram of the elution and amplification of captured DNA fragments in a method of the present invention.

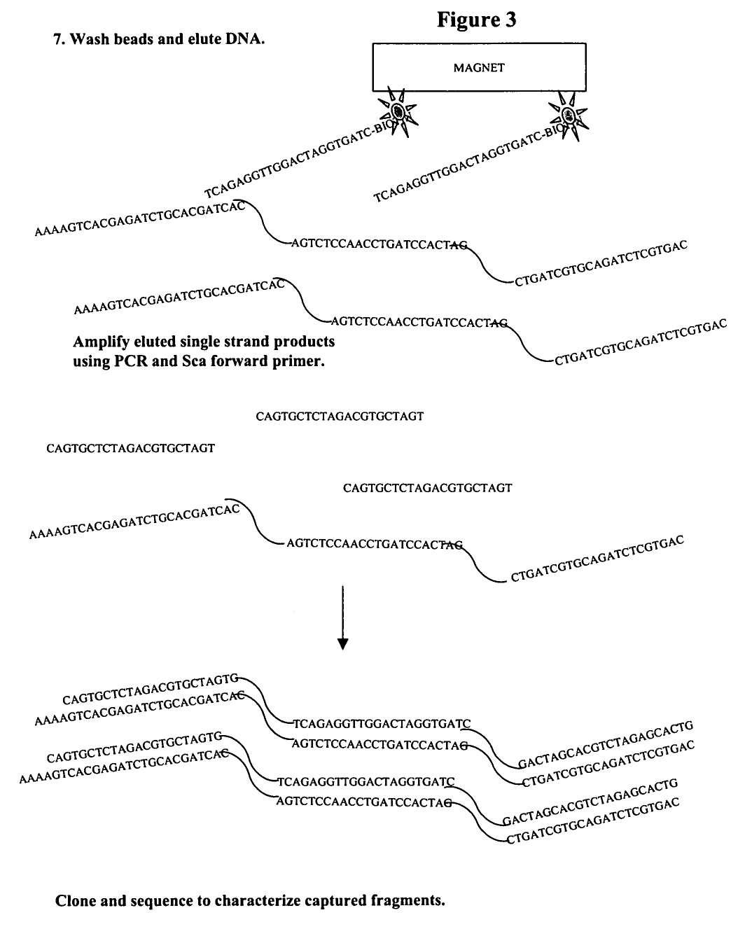

### DETAILED DESCRIPTION OF THE INVENTION

The present invention provides a method of isolating a nucleic acid molecule of interest when at least a partial sequence of the target nucleic acid molecule is known. Efficient isolation of specific nucleic acid targets allows for the capture of any nucleic acid targets. The method represents a significant improvement in efficiency and can be adapted to the isolation of a wide variety of genomic targets including but not limited to microsatellites. The method includes hybridizing nucleic acid fragments to a functionalized nucleic acid probe. The functionalized nucleic acid probe is then complexed with a capture agent which can, in turn, be immobilized thereby immobilizing the nucleic acid molecule of interest that is hybridized to the functionalized probe. This nucleic acid molecule of interest is then eluted from the functionalized nucleic acid probe.

In the first step of the method of the present invention, a nucleic acid probe is hybridized to the target nucleic acid fragment. The nucleic acid probe used in this step must be specifically designed to recognize and bind to the target nucleic acid and be functionalized to incorporate a label that will complex with a capture agent in subsequent steps of the methods of the present invention.

To hybridize with efficiency, the nucleic acid probe must have a sequence that is complimentary to at least a portion of the target nucleic acid molecule. The efficient isolation of specific nucleic acid targets allows for the capture of any desired segment of DNA or cDNA. A probe can be designed for any specific nucleic acid target. The nucleic acid targets may have sequences of either high complexity or low complexity. For the purposes of this disclosure, a high complexity nucleic acid sequence is a nucleic acid sequence having no sequences of less than 10 consecutive base pairs that repeat within the target nucleic acid. Examples of low complexity nucleic acid targets include microsatellites scattered throughout the genome of an organism. One of skill in the art will readily appreciate that the required partial sequence may be obtained from a wide variety of sources. Examples include references disclosing nucleic acid sequences that overlap the target nucleic acid sequence, known flanking sequences of the nucleic acid of interest, partial sequences of nucleic acids that are related to the target nucleic acid by alternative splicing, the coding region of functional protein domains known or believed to be present in a protein encoded by the nucleic acid of interest. Alternatively, a “degenerate” nucleic acid sequence may be compiled from the amino acid sequence of a protein known to be encoded by the target nucleic acid sequence. Typically, the probe sequence is designed by alignment of a highly conserved region or regions of the corresponding known nucleic acid sequence from other species. The probe used is chosen by the operator according to the selected target. The melting temperature of all probes should be below 70° C. to maintain the integrity of the components of this process.

The functionalization of the probe can also take many forms. The only requirement is that the functional group selectively interact with a corresponding capture agent such that the probe, and any target nucleic acid hybridized to it, can be isolated from a sample of unrelated biological molecules including other nucleic acid molecules unrelated to the target nucleic acid. Additionally, the functional group must not prevent or severely inhibit hybridization of the probe to the target nucleic acid. Examples of useful groups for functionalization of probes include small proteins recognized by specific antibody capture agents, metalloporphyrins that can be attracted by magnetic capture agents, and biotin vitamin or avidin proteins that recognize and bind to one another with high affinity.

In a preferred embodiment of the present invention, the functional group is a biotin label attached to the 3′ end of the probe. This functionalization also serves to block extension of the probe in later reactions. The functionalization of this probe is completed when streptavidin coated magnetic beads are added to the reactants and bind to the biotin linked to the probe. Streptavidin coated magnetic beads aid in the separation of the DNA fragments containing the target sequence from the remaining fragments in the solution. Streptavidin bonds with very high affinity to biotin that in turn is covalently bound to the probe. The streptavidin coated magnetic beads are preferably added to the nucleic acid sequences after the hybridization step. This allows for the biotin-streptavidin complex to form while preventing interference of the streptavidin coated magnetic beads with hybridization between the biotin labeled probe and the target nucleic acid molecules. Preferably, the streptavidin coated magnetic beads are treated with a blocking agent to reduce non-specific binding (background) during the capture step described below. The blocking agent may include any of the known blocking agents available in the art such as protein blocking agents or heterologous DNA, for example, salmon sperm DNA. Preferably, the blocking agent is a protein blocking agent as the protein-based blocking materials reduce the isolation of unrelated and nonspecific nucleic acid molecules and increase the successful isolation of the target nucleic acid. The use of a protein-based blocking material increases the isolation of target nucleic acids (as opposed to unrelated nucleic acid molecules) by about ten-fold over the use of salmon sperm DNA. Streptavidin magnetic beads are available commercially and are prepared by several washes in buffer followed by incubation with protein based blocking materials. The incubation is typically conducted at room temperature on a rocker platform followed by several additional washes and resuspension in a buffer compatible with the buffer used for hybridization of the functionalized probe to the nucleic acid fragments.

The target nucleic acid may be isolated from a wide variety of sources. Typically, the target nucleic acids are isolated from biological samples containing the nucleic acid sequences of interest as well as other biological molecules that may include unrelated nucleic acid molecules. Preferably, the target nucleic acid fragments are isolated from genomic or cDNA fragments composed of fragmented DNA from one or more individuals suspected of harboring the sequence of interest. If the target nucleic acid is an RNA molecule, reverse transcriptase is employed to convert RNA into cDNA for the gene expression studies. For enrichment studies, genomic DNA from one or more individuals of the targeted species is pooled to allow for random sampling. But it is not necessary to use DNA pooled from several individuals, and in the case of gene expression studies, pooling DNA should be avoided. Additionally, the reaction can be scaled down to accommodate samples with low DNA concentrations.

Depending on the size of the DNA fragments within the biological samples in which the target nucleic acid fragments reside, the DNA may be first digested with different restriction nuclease enzymes. The enzymatic digestion of the DNA can be altered to decrease or increase the size of the DNA fragments recovered from this method. This allows for selection of DNA fragments in any size range. Additional enzymes can be added if smaller fragments are desired. Conversely, a restriction enzyme that cuts at fewer recognition sites can be substituted for another restriction endonuclease or eliminated if larger fragments are required. In the case of cDNA, it may not be necessary to use endonucleases if the cDNA sizes are within a desired range. Additionally, if the biological sample containing the nucleic acid molecules contains many other nonspecific biological molecules that may interfere with the hybridization, the sample may optionally be treated to enrich the nucleic acid molecules while reducing or eliminating the nonspecific molecules in the sample. Many enrichment or isolation procedures known in the art are suitable to prepare the nucleic acid fragments for use in the present invention.

The use of different probes dictates the need to change the hybridization temperature due to the differences in the melting temperatures between probes. Typically, the hybridization temperature should be between about 5° C. and about 10° C. below the melting temperature of the probe. The fragmented DNA is hybridized to the functionalized probe in the presence of a biologically compatible buffer. Preferably, the hybridization is performed in 6×SSC. For example, the reactants can be combined by adding about 100 ng DNA and about 100 pmol probe are added to 10×SSC (1.5M NaCl, 0.15M Na₃C₆H₅O₇.2H₂O) and water. The reactants are heated to well above the melting temperature of the probe and then cooled to allow for hybridization. For example, the reactants are typically heated to about 95° C. for about 10 minutes and incubated at a temperature of between about 5° C. to about 10° C. less than the melting temperature of the probe for about 1 hour. After the hybridization step, the probe is now bound by hydrogen bonding to nucleic acid fragments that contain the complementary target nucleic acid sequence.

In a preferred embodiment of the present invention, DNA linkers are ligated to the ends of the nucleic acid fragments prior to hybridization with the functionalized probe. These linkers are short strands of DNA that can serve as linkers for subcloning of the target nucleic acid sequences following hybridization and subsequent isolation. Additionally, after ligation, these linkers present a short strand of known DNA sequence flanking at least one side of the target nucleic acid sequence. Therefore, these linkers can hybridize with DNA primers for priming DNA sequencing and PCR amplifications of the isolated target nucleic acid sequences. The hybridization occurs between the functionalized probe and the linker ligated fragments. In some cases the efficiency of the method is increased by using linker ligated fragments that have been amplified by the polymerase chain reaction using a primer having a sequence complementary to a linker such that the target nucleic acid fragment that hybridizes to the probe will be amplified prior to the hybridization. This is especially useful when working with low copy number targets or low DNA concentrations.

The linkers can be designed to have overhanging ends that correspond in sequence to the cut sight of a restriction nuclease enzyme or they may be designed with blunt ends if the fragmented DNA is to be digested with an exonuclease to leave blunt ended DNA fragments for ligation.

To use linkers in the method of the present invention, the biological sample containing the target nucleic acid sequences is digested for a sufficient length of time under conditions sufficient to fragment the majority of nucleic acid molecules present in the sample. The nucleic acid fragments are then ligated to the linkers or further digested with an exonuclease to form blunt ended fragments followed by ligation of blunt ended DNA strands. Typically, the biological sample containing the nucleic acid is digested in the presence of one or more restriction endonucleases that function in the same or similar salt conditions at 37° C. for a period of between about 1 hour and about 24 hours. Following the digestion, the reactants are heated to about 65° C. for about 20 minutes to denature the restriction nucleases and stop the digestion reaction.

In a particularly preferred embodiment of the present invention, the restriction endonucleases and the linkers ligated to the ends of the fragmented nucleic acid molecules are specifically designed to function together. For example, the DNA sequence of the linkers can incorporate part of the sequence recognized by one or more of the restriction nucleases used to fragment the nucleic acids such that overhanging ends on the linkers have the complementary sequence to the overhanging ends of the fragmented nucleic acid sequences. This design can greatly increase the efficiency of ligation of the linkers and, if designed correctly, can allow for directional cloning of the target nucleic acid sequences following hybridization and isolation. Alternatively, the linkers can be designed to incorporate the recognition sequence of a restriction endonuclease that makes a blunt end cut of the primer that is subsequently ligated to the end of the nucleic acid fragments that have been treated with an exonuclease to leave a blunt end.

An example of a combination of restriction endonucleases and linkers designed to function together that is well suited for use in the methods of the present invention includes fragmentation of the nucleic acid with the Csp6 I and Xma I restriction endonucleases. Csp6 I recognizes and cleaves the four bp sequence 5′-GTAC-3′ while Xma I is a six base pair cutter recognizing the sequence 5′-CCCGGG-3′. Both enzymes result in a 5′ overhang.

The 5′ overhangs are removed by the digestion with mung bean exonuclease followed by dephosphorylation. The blunt ended nucleic acid fragments are then ligated, in the presence of the Sca I restriction endonuclease, to linkers having the following sequences:

| | |
|---|---|
| 5′-CAGTGCTCTAGACGTGCTAGT-3′ | (SEQ ID NO:1) |
| 5′-ACTAGCACGTCTAGAGCACTGAAAA-3′. | (SEQ ID NO:2) |

These linkers are formed by the action of the Sca I restriction endonuclease on a double stranded DNA molecule with one Sca I cut site that results in the formation of two identical double-stranded linkers each with a 3′ poly A overhang having the sequences shown in FIG. 4, in which the blunt ended Sca I cut site is between the A and T bases at the position indicated by the arrow heads. The annealed product is a double stranded linker on which one end is blunt while the other has a 3′ overhang to decrease the formation of linker dimers. Additionally, the reverse linker is phosphorylated at the 5′ base during manufacturing. Each blunt end contains half the recognition sequence for the enzyme Sca I (a blunt-end, 6 bp cutter that cleaves 5′-AGTACT-3′). When blunt ends come together to form a dimer, the Sca I site is restored. Thus, ligation of these linkers in the presence of the Sca I restriction endonuclease further prevents the formation of primer dimers and increases the efficiency of the blunt end ligation of the linkers to the nucleic acid fragments.

Since the formation of linker dimers results in the restoration of the Sca I recognition site, the addition of the Sca I enzyme to the ligation reaction serves to cleave linker dimers. This keeps the linkers available for ligation to the nucleic acid fragments. The use of Csp6 I in the DNA digestion arrests the ability of Sca I to further cleave the DNA. Csp6 I cleaves a sequence, 5′-GTAC-3′, internal to the Sca I site, (5′-AGTACT-3′). The overhang produced by the Csp6 I digestion is digested with the mung bean exonuclease; thus, all sites for Csp6 I and Sca I are destroyed. The robustness of the linker ligation reaction can be monitored by polymerase chain reaction (PCR) using the forward linker (SEQ ID NO: 1) only as the primer.

Following the hybridization of the target nucleic acid fragments to the functionalized probe, the probe is complexed with a capture agent. Because the functionalized probe is hybridized to the target nucleic acid fragment, the complex of the capture agent and the probe includes the target nucleic acid fragment. Therefore, this step of complexing the probe necessarily includes complexation of the target nucleic acid fragments within the biological sample.

The capture agent can be any entity that interacts selectively with the chosen functional agent linked to the probe. For example, if the probe was functionalized by the attachment of a specific protein, the capture agent may be an antibody recognizing the protein. Conversely, if the probe was functionalized with an antibody, or a functional part thereof, the capture agent may be a protein recognized by the antibody. Similarly, the capture agent and the functional agent linked to the probe may be combinations of organic or inorganic molecules with strong affinity for one another including, but not limited to, biotin and steptavadin, magnets and metals or molecules incorporating metals, or proteins and antibodies. Preferably, the combination includes biotin and streptavidin. More preferably, the probe is functionalized with at least one biotin molecule which is bound to streptavidin-coated magnetic particles and the capture agent is a magnet. In one embodiment of the present invention based on this combination, the streptavidin coated magnetic beads, bound biotin labeled probe and the hybridized fragments are captured within 30 to 45 seconds at room temperature using a magnetic stand.

Following this capture, the captured probes and hybridized DNA fragments may be washed. Preferably, this wash continues through progressively more stringent washes until the target DNA strands are essentially free of any nonspecific biological molecules that are not hybridized to the probe. Changing the wash temperatures acts to increase or decrease the stringency of the procedure. The final wash temperature preferably ranges from about 4° C. to about 7° C. below the hybridization temperature. Preferably, the washes include two each of 2×SSC and 1×SSC at room temperature followed by two washes of 1×SSC at about 50° C. Each wash entails the addition of wash buffer and the resuspension of the hybridized probes in the wash buffer by gently agitating the tube.

In the embodiment of the present invention in which a magnetic molecule is used to functionalize the probe and the capture agent is a magnet, the magnet can be applied after the washes to separate the probes and associated fragments from the wash buffer.

After the hybridized probe has been isolated from the biological sample through complexation with the capture agent, the target nucleic acid sequence is eluted from the probe to leave the target nucleic acid fragment isolated from the biological sample for further study. The elution of the nucleic acid fragments from the probe is dependent on the melting temperature of the probe. The elution is performed under conditions that will cause the hydrogen bonds formed between the probe and the target nucleic acid fragments to be denatured. The elution is conducted in water and the temperature of the elution should be at or just above the melting temperature of the probe. Because no salts are available in this elution to stabilize the hydrogen bonds between the probe and the fragment, increasing the temperature substantially above the melting temperature will not increase the yield. However, in the embodiment in which magnetic beads are used in the capture agent or the functionalization of the probe, an increase in an elution temperature above about 70° C. may degrade the magnetic beads and interfere with subsequent isolation steps. For example, the addition of water and a subsequent incubation at about 65° C. for about 5 minutes denatures the hydrogen bonds formed releasing the fragments from the probe. The magnetic stand is used to separate the beads and bound probe from the target DNA fragments that are transferred to a fresh tube. In the embodiment of the present invention in which the probe is functionalized with a magnetic molecule and a magnet is employed as the capture agent, the magnet may then be used to separate the beads and bound probe from the target nucleic acid fragments.

The single-stranded isolated target nucleic acid fragments are then available for further study and characterization. Typically, the first step in this characterization is formation of the complementary strand. This can be accomplished with any of the well known methods in the art. For example random primers or primers designed from known sequence within the target nucleic acid fragments can be hybridized to the single-stranded isolated target nucleic acid fragments and extended with a DNA polymerase enzyme.

If linkers were ligated to the ends of the target nucleic acid fragments in the embodiment of the present invention described above, primers designed to hybridize to the known sequence of the linkers can be used in conjunction with a DNA polymerase to prime and extend the complementary strand. Alternatively, in this embodiment of the present invention, primers complementary to the ligated linker sequences can be used to form the complementary strand and amplify the single-stranded isolated target nucleic acid fragments in the polymerase chain reaction. PCR amplification generates ample double stranded product for cloning.

Having produced the complementary strand and optionally amplified the isolated nucleic acid fragments, the fragments can be cloned and sequenced to allow for further characterization. The fragments are ligated and transformed using standard procedures and the recovered products are sequenced by conventional methods.

Additional objects, advantages, and novel features of this invention will become apparent to those skilled in the art upon examination of the following examples thereof, which are not intended to be limiting.

### EXAMPLES

### Example 1

This example illustrates the isolation of CR1 transposable elements, a somewhat elusive retrotransposon. As one of skill in the art will readily appreciate, the following methodology can be customized for the isolation of other target nucleic acid sequences of interest by simply substituting the appropriate probe sequence.

A. DNA Digestion

Genomic DNA from one or more individuals of the targeted species is pooled to allow for random sampling. Ten micrograms of the pooled DNA is fragmented in a 100 μl double restriction endonuclease digestion using 5 μl Csp6 I (10,000 U/ml, Fermentas), 5 μl Xma I (10,000 U/ml, New England Biolabs (NEB)), 10 μl 10×BSA (NEB), 10 μl 10×NEB buffer 2 and H₂O to 100 μl. The reaction is incubated overnight at 37° C. The majority of resulting fragments range in size from 300 to 1200 base pairs (bp). Csp6 I recognizes and cleaves the four bp sequence 5′-GTAC-3′ while Xma I is a six base pair cutter recognizing the sequence 5′-CCCGGG-3′. Both enzymes result in a 5′ overhang. After incubation the digest reaction is heated for 20 minutes at 65° C. to denature the enzymes.

B. Digest Overhangs with Mung Bean Exonuclease.

The 5′ overhangs were removed by the addition of 1 μl of mung bean exonuclease (NEB) directly to the 100 μl digest reaction followed by a 45 minute incubation at 30° C. The 100 μl reaction containing the blunt ended digested fragments is purified using the Qiaquick PCR purification kit (Qiagen) following manufacturer's protocol. The DNA was eluted in 50 μl kit EB buffer. To dephosphorylate the fragments, 6 μl NEB buffer 2, 3 μl H₂O and 1 μl calf intestinal phosphatase (10,000 U/ml, CIP, NEB) was added to the 50 μl eluted DNA. The reaction takes place at 37° C. for 2 hours. The dephosphorylation of the fragments increases the efficiency of the following linker ligation reaction by inhibiting any ligation of the fragments to each other. In a post-dephosphorylation Qiaquick PCR purification kit clean up, the DNA is eluted in 30 μl EB buffer.

C. Ligate Sca Linkers in the Presence of Sca 1.

The blunt ended dephosphorylated fragments were ready for linker ligation. The Sca linkers are prepared using two oligonucleotides that are designated by convention as the Sca forward and Sca reverse linker. The Sca forward linker sequence is:

| | |
|---|---|
| 5′-CAGTGCTCTAGACGTGCTAGT-3′ | (SEQ ID NO.: 1) |

while the reverse Sca linker contains the sequence:

| | |
|---|---|
| 5′-ACTAGCACGTCTAGAGCACTGAAAA-3′. | (SEQ ID NO.: 2) |

The forward and reverse linkers were annealed by heating an equal volume of 10 μM linkers (in H₂O) for 5 minutes at 94° C. followed by a room temperature incubation for 10 minutes resulting in 5 μM Sca linker.

Annealed linkers were ligated to the DNA fragments in a 30 μl reaction containing 11.7 μl 5 μM double stranded linkers, 3 μl NEB buffer 2, 3 μl 10 mM rATP, 0.3 μl 100×BSA, 10 μl DNA and 1 μl each Sca I restriction endonuclease (10,000 U/ml, NEB) and T4 DNA ligase (2×10⁶ U/ml, NEB). The reaction proceeded overnight (18 hours) cycling from 16° C. for 30 minutes to 37° C. for 10 minutes.

D. Hybridize Fragments to a Biotin Labeled Probe.

In this example, the CR1COSUTR-B probe was used to capture DNA fragments containing the CR1 transposable element. The probe sequence:

| | |
|---|---|
| 5′-TCAGAGGTTGGACTAGGTGATC-3′ | (SEQ ID NO.: 5) |

was designed from an alignment of the highly conserved 3′ untranslated region (UTR) of CR1 elements from chicken, turtle and coscoroba. The probe used was chosen by the operator according to the selected target with the requirement that the melting temperature not exceed 70° C. The required biotin label is placed on the 3′ end of the probe. This blocked extension of the probe in later reactions.

The prepared fragmented DNA was hybridized to the biotin labeled probe in the presence of 6×SSC. Approximately 100 ng DNA (2 μl) and 100 pmol of 50 μM probe (2 μl) were added to 60 μl 10×SSC (1.5M NaCl, 0.15M Na₃C₆H₅O₇.2H₂O) and 36 μl H₂O. The reaction was heated to 95° C. for 10 minutes and incubated at 55° C. for 1 hour.

E. Add Blocked Streptavidin Coated Magnetic Beads.

Streptavidin coated magnetic beads aid in the separation of the DNA fragments containing the target sequence from the remaining fragments in the solution. Streptavidin bonds with very high affinity to biotin that in turn is covalently bound to the probe. After the hybridization step, the probe is bound by hydrogen bonding to the linker ligated DNA fragments that contain the complementary target sequence. 100 μl streptavidin magnetic beads (Promega) were washed three times with 100 μl 6×SSC prior to the addition of 100 μl bead block buffer (0.2% I block reagent (Tropix), 0.5% sodium dodecylsulfate (SDS) in PBS (0.058M Na₂HPO₄, 0.017M NaH₂PO₄.H₂O, 0.068M NaCl). The blocking solution and beads were incubated for 45 minutes at room temperature on a rocker platform. Three washes with 100 μl 6×SSC follow the bead block and the blocked beads were resuspended in 100 μl 6×SSC.

F. Magnetic Capture the Magnetic Beads, Biotin Labeled Probe and Associated Fragments.

The 100 μl of pretreated beads were added to the 100 μl hybridization reaction and incubated at the room temperature for 10 minutes with occasional mixing. The beads, bound biotin labeled probe and the corresponding fragments were captured within 30 to 45 seconds at room temperature using a magnetic stand (Promega) followed by a series of six washes described below.

G. Wash Beads and Elute DNA.

The washes included two each of 200 μl 2×SSC and 1×SSC at room temperature followed by two washes of 200 μl 1×SSC at 50° C. Each wash entailed the addition of 200 μl wash buffer and the resuspension of the beads in the wash buffer by gently flicking the tube. Applying the magnet separated the beads and associated fragments from the wash buffer. The addition of 50 μl H₂O and a subsequent incubation at 65° C. for 5 minutes denatured the hydrogen bonds formed between the probe and the DNA fragments releasing the fragments from the probe. The magnetic stand was used to separate the beads and bound probe from the target DNA fragments that were transferred to a fresh tube.

H. Amplify Eluted Single Strand Products Using PCR and the Sca Forward Primer.

At this stage, the known linkers that flank the partially known, single stranded target DNA fragments aid in the production of the complementary strand. PCR amplification generates ample double stranded product for cloning. The 50 μl PCR reaction includes 5 μl 10× Thermopol buffer (NEB), 5 μl 8 mM dNTPs, 4 μl 10 μM Sca forward primer, 25.7 μl H₂O, 10 μl eluted DNA and 0.3 μl Vent exo⁻ polymerase (2,000 U/ml, NEB). The reaction profile began with a 5 minute 95° C. denaturing step followed by 30 cycles of 95° C. for 45 seconds, 58° C. for 1 minute and 72° C. for 2 minutes. A 10 minute extension step concluded the reaction. Running more than 30 cycles appeared to increase the background and is therefore not recommended. The PCR product was electrophoresed on a 1% agarose gel containing 0.1% gel star (Cambrex) and the resulting smear was quantified by comparing the smear intensity to the intensity of a known quantity of marker.

I. Clone and Sequence to Characterize Captured Fragments.

Ligation and transformation were performed following Strategene's PCR-Script Amp cloning kit protocol using the post hybridization PCR product. The column provided with the kit was used to clean up the PCR product and the purified product was released from the column in 50 μl H₂O. The ligation into the kit vector requires a proper insert to vector ratio. The amount of product may be low and diluting the vector by 20% with H₂O can aid in obtaining the correct ratio. The use of Xma1 in the original DNA digest eliminated further digestion of the fragments by the kit supplied enzyme, Srf 1. Xma 1 recognizes and cleaves a sequence internal to the Srf 1 site and this essentially destroys all Srf 1 sites in the fragments. The transformation proceeded following the kit protocol. The transformed cells were plated onto S-Gal/IPTG (Sigma) ampicillin plates and incubated overnight at 37° C.

White colonies were selected, individually lifted with a sterile pipet tip and placed in 100 μl T.E (10 mM Tris pH 8.0, 0.1 mM EDTA). The colonies were heated to 100° C. for 10 minutes and vortexed briefly. One microliter of the 100 μl colony touch was used as the template in a 25 μl PCR reaction with 1 μM each T7 and T3 primers using a reaction mix containing 250 μM each dNTP, 0.63U Taq polymerase (Promega) in 1×Taq buffer (67 mM Tris.HCl pH 8.0, 6.7 mM MgSO₄, 16.6 mM (NH₄)₂SO₄, 10 mM B-mercaptoethanol). A 94° C. preheat for 2 minutes was followed by 30 cycles of 94° C. for 40 seconds, 60° C. for 90 seconds and 72° C. for 2 minutes. A 10 minute post heat at 72° C. concluded the reaction. The products were sized by electrophoresis on a 1% agarose gel containing 0.5 μg/ml ethidium bromide. Products are sequenced by conventional methods.

### Example 2

A study was conducted on an invertebrate (snail) to demonstrate the robustness of the method of the present invention. Although the invention was initially designed using vertebrates, a variety of microsatellites was rapidly isolated from this entirely new phylum on the initial attempt.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of isolating a high complexity nucleic acid molecule comprising:
a. hybridizing high complexity nucleic acid fragments to a functionalized nucleic acid probe having a sequence complimentary to at least a portion of a high complexity nucleic acid molecule to form hybridized nucleic acid fragments;
b. complexing the functionalized nucleic acid probe with a capture agent;
c. immobilizing the capture agent; and,
d. eluting the high complexity nucleic acid molecules from the functionalized nucleic acid probe.

2. The method of claim 1, wherein the functionalized nucleic acid probe is a biotinylated nucleic acid probe.

3. The method of claim 2, wherein the hybridizing step comprises incubating the high complexity nucleic acid fragments with a biotinylated nucleic acid probe at a temperature of between about 45° C. and about 70° C. for about 1 hour.

4. The method of claim 1, wherein the capture agent comprises streptavidin-coated magnetic beads.

5. The method of claim 1, wherein the streptavidin-coated magnetic beads comprise a protein-blocking material.

6. The method of claim 1, comprising the additional step of ligating at least one DNA linker to the ends of digested high complexity nucleic acid fragments to form ligated nucleic acid fragments prior to the hybridizing step.

7. The method of claim 6, wherein the DNA linker comprises an oligodeoxynucleotide having the sequence of SEQ ID NO:1 and an oligodeoxynucleotide having the sequence of SEQ ID NO:2 which together form the DNA linker.

8. The method of claim 6, wherein the digested high complexity nucleic acid fragments are produced by incubating a nucleic acid with a nuclease enzyme selected from the group consisting of Csp6, Xba I, mung bean exonuclease, Sca I and combinations thereof.

9. The method of claim 6, wherein the ligating step takes place in the presence of Sca I endonuclease.

10. The method of claim 1, wherein the eluting step comprises:
a. washing the magnetic beads with a wash buffer at about 50° C.; and,
b. incubating the magnetic beads in water at about 65° C.

11. The method of claim 1, comprising the additional steps of:
a. amplifying the isolated high complexity nucleic acid fragment; and,
b. sequencing the amplified high complexity nucleic acid fragment.

12. The method of claim 11, comprising the additional step of ligating at least one DNA linker to the ends of digested high complexity nucleic acid fragments to form ligated nucleic acid fragments prior to the hybridizing step, and wherein the amplification step utilizes a DNA primer having a sequence complementary to one strand of the linker.

13. The method of claim 12, wherein the amplification step comprises the polymerase chain reaction.

14. The method of claim 12, wherein the amplification step comprises:
a. ligating the isolated high complexity nucleic acid fragment into a vector;
b. transforming the ligated vector into a microorganism;
c. amplifying the vector by maintaining the microorganism under conditions favoring growth of the microorganism; and,
d. recovering the amplified vector from the microorganism.

15. A kit for isolation of a nucleic acid fragment comprising:
a. DNA linkers comprising an oligodeoxynucleotide having the sequence of SEQ ID NO:1 and an oligodeoxynucleotide having the sequence set forth in SEQ ID NO:2 which together form the DNA linker,
b. streptavidin-coated magnetic beads, and
c. a protein blocking material.

16. The kit of claim 15, comprising additional components selected from the group consisting of instruction manual, buffers, nucleases, wash solution concentrates, PCR primers, PCR buffers, Taq polymerase, PCR product isolation columns and combinations thereof.

17. A DNA linker comprising an oligodeoxynucleotide having the sequence of SEQ ID NO:1 and an oligodeoxynucleotide having the sequence of SEQ ID NO:2 which together form the DNA linker.

18. A DNA primer comprising the sequence of SEQ ID NO: 1.

19. A method of isolating a nucleic acid molecule comprising:
a. ligating at least one DNA linker to digested nucleic acid fragments, wherein said linker is formed by an oligodeoxynucleotide having the sequence of SEQ ID NO:1 and an oligodeoxynucleotide having the sequence of SEQ ID NO:2;
b. hybridizing the nucleic acid fragments to a biotinylated nucleic acid probe having a sequence complimentary to at least a portion of the nucleic acid molecule;
c. complexing the biotinylated nucleic acid probe with streptavidin-coated magnetic beads comprising a protein-blocking material;
d. immobilizing the streptavidin-coated magnetic beads with a magnet; and,
e. eluting the nucleic acid molecules from the biotinylated nucleic acid probe.

20. The method of claim 19, wherein the hybridizing step comprises incubating the nucleic acid fragments with the biotinylated nucleic acid probe at a temperature of less than about 70° C. for about 1 hour.

21. The method of claim 19, wherein the digested nucleic acid fragments are produced by incubating a nucleic acid with a nuclease enzyme selected from the group consisting of Csp6I, Xba I, mung bean exonuclease, Sca I and combinations thereof.

22. The method of claim 19, wherein the ligating step takes place in the presence of Sca I endonuclease.

23. The method of claim 19, wherein the eluting step comprises:
a. washing the magnetic beads with a wash buffer at about 50° C.; and,
b. incubating the magnetic beads in water at about 65° C.

24. The method of claim 19, comprising the additional steps of:
a. amplifying the isolated nucleic acid fragment; and,
b. sequencing the amplified nucleic acid fragment.

25. The method of claim 24, wherein the amplification step comprises the polymerase chain reaction.

26. The method of claim 24, wherein the amplification step comprises:
a. ligating the isolated nucleic acid fragment into a vector;
b. transforming the ligated vector into a microorganism;
c. amplifying the vector by maintaining the microorganism under conditions favoring growth of the microorganism; and,
d. recovering the amplified vector from the microorganism.
